(19) United States
(12) Patent Application Publication
(10) Pub. No.: US 2009/0240214 A1
(43) Pub. Date: Sep. 24, 2009

(54) CATHETER WETTING SYSTEM AND METHOD

(76) Inventors: Anthony J. Conway, Chatfield, MN (US); Richard D. Fryer, JR., Chatfield, MN (US)

Correspondence Address: MERCHANT & GOULD PC, P.O. BOX 2903, MINNEAPOLIS, MN, 55402-0903, US

(21) Appl. No.: 12/051,119

(22) Filed: Mar. 19, 2008

(51) Int. Cl. A61B 19/02; A61M 25/00

(52) U.S. Cl. 604/265; 206/572

(57) ABSTRACT

The present invention includes a system and method for wetting a catheter. The wetting system can include a catheter container, a fluid packet, and a catheter. The fluid packet and catheter container are adapted and configured to require opening the packet and moving the container to wet the catheter. The method includes opening the packet and moving the container to wet the catheter.

### FIELD OF THE INVENTION

The present invention includes a system and method for wetting a catheter. The wetting system can include a catheter container, a fluid packet, and a catheter. The fluid packet and catheter container are adapted and configured to require opening the packet and moving the container to wet the catheter. The method includes opening the packet and moving the container to wet the catheter.

### BACKGROUND OF THE INVENTION

Wetting of wettable catheters, such as hydrophilic urinary catheters, produces lubrication that is advantageous for insertion into the urethra. The lubrication is increases the comfort of the catheterized subject and eases insertion of the catheter. Inserting a dry or incompletely wetted catheter can be painful.

Wettable catheters, particularly hydrophilic urinary catheters, can be wetted by a variety of methods. For example, the catheter can be removed from its package and wetted. Such a method of wetting risks contaminating the catheter. Alternatively, some catheters are packaged with a container of fluid and opening the fluid container is sufficient to wet the insertable length of the catheter. Such a package is complicated and requires a large volume of fluid, which must be disposed of.

There remains a need for additional systems and methods for wetting wettable catheters.

### SUMMARY OF THE INVENTION

The present invention includes a system and method for wetting a catheter. The wetting system can include a catheter container, a fluid packet, and a catheter. The fluid packet and catheter container are adapted and configured to require opening the packet and moving the container to wet the catheter. The method includes opening the packet and moving the container to wet the catheter.

In an embodiment, the wetting system includes a catheter container adapted and configured to enclose the fluid packet and the catheter. The fluid packet is adapted and configured to be opened and to release fluid without opening the catheter container. The fluid packet is adapted and configured to be squeezed, pressed, or struck to release fluid. The fluid packet and the catheter container are adapted and configured so that mere release of fluid from the fluid packet only incompletely wets the catheter. Mere release of fluid from the packet does not wet the catheter to an extent sufficient to render the catheter insertable. The system is adapted and configured for movement or manipulation of the container to wet the catheter to an extent that it can be inserted.

### BRIEF DESCRIPTION OF THE DRAWINGS

FIG. 1 schematically illustrates an embodiment of the present system including a catheter pouch, a fluid packet, and a wettable catheter.

FIG. 2 schematically illustrates the embodiment of FIG. 1, in which the fluid has been released from the packet, but the pouch has not been moved.

FIG. 3 schematically illustrates the embodiment of FIG. 2, in which the pouch has been moved to wet the coated surface of the wettable catheter.

FIG. 4 schematically illustrates an embodiment of the present system including a catheter pouch in the form of a segmented catheter pouch, a fluid packet, and a wettable catheter.

FIG. 5 schematically illustrates the embodiment of FIG. 4, in which the fluid has been released from the packet, but the pouch has not been moved.

FIG. 6 schematically illustrates the embodiment of FIG. 5, in which the pouch has been moved to wet the coated surface of the wettable catheter.

### DETAILED DESCRIPTION OF THE INVENTION

### Definitions

As used herein, the phrase “hydrophilic catheter” refers to a catheter on which the shaft and tip have a hydrophilic surface that when wet that provides advantageous lubrication during insertion and use of the hydrophilic catheter.

As used herein, the phrase “hydrophilic urinary catheter” refers to a hydrophilic catheter sized and constructed for occupying a mammalian urethra and draining a mammalian bladder.

As used herein, the phrase “insertable length” of a hydrophilic urinary catheter refers to the length of the catheter shaft that is coated with the hydrophilic material and inserted into the subject's urethra. For a human female subject, the insertable length is about 80-140 mm. For a human male subject, the insertable length is about 200-350 mm.

As used herein, the phrases “only incompletely wets”, “incompletely wetting”, and like phrases refer to wetting less than the insertable or inserted length of the catheter and/or not wetting the insertable or inserted length of the catheter to a sufficient extent to render it insertable into a human urethra without unacceptable discomfort. For example, the entire insertable length of the catheter can be wetted, but not wetted to a sufficient extent to render it insertable. Wetting to a sufficient extent requires contact with sufficient wetting fluid to wet the catheter for a sufficient time to wet the catheter. For example, a wettable catheter can include a coating that requires wetting for insertion of the catheter. Each unit volume of the coating can require contact with sufficient wetting fluid to wet that entire coating volume. Each unit volume of the coating can require contact with wetting fluid for sufficient time for the wetting fluid to wet that entire coating volume. For example, contact with visible liquid water for about 10, about 20, to 30 seconds or more can wet the coating on a hydrophilic urinary catheter.

### Catheter Wetting System and Method

The present invention relates to a system and method for wetting a catheter. The system can include a catheter, a catheter container, and a fluid packet. The system can be employed in the method of the invention.

Catheters suitable for the system and method include catheters that benefit from wetting before use, for example, before insertion into a subject. Such a catheter is referred to herein as a wettable catheter. Wettable catheters include hydrophilic catheters, such as hydrophilic urinary catheters.

In the present system and method, the catheter container and the fluid packet are configured so that opening the fluid packet in the catheter container only incompletely wets the catheter. For example, the catheter container can be configured so that fluid does not move readily along the length of the catheter unless the catheter container is moved. For example, the catheter can be positioned within the catheter container so that fluid does not move readily along the length of the catheter unless the catheter container is moved. For example, the fluid packet can contain an amount of fluid that does not readily move along the length of the catheter unless the catheter container is moved. For example, the fluid packet can be positioned so that fluid that does not readily move along the length of the catheter unless the catheter container is moved.

For example, the catheter container can be configured so that it must be moved to contact the catheter with sufficient wetting fluid for a sufficient time to completely wet the catheter. For example, the catheter can be positioned within the catheter container so that the container must be moved to contact the catheter with sufficient wetting fluid for a sufficient time to completely wet the catheter. For example, the fluid packet can contain an amount of fluid so that the container must be moved to contact the catheter with sufficient wetting fluid for a sufficient time to completely wet the catheter. For example, the fluid packet can be positioned within the catheter container so that the container must be moved to contact the catheter with sufficient wetting fluid for a sufficient time to completely wet the catheter.

For example, the catheter container can be configured so that it must be moved to contact the insertable length of the catheter with wetting fluid. For example, the catheter can be positioned within the catheter container so that the container must be moved to contact the insertable length of the catheter with wetting fluid. For example, the catheter can be positioned within the catheter container so that the container must be moved to contact the insertable length of the catheter with wetting fluid. For example, the fluid packet can contain an amount of fluid so that the container must be moved to contact the insertable length of the catheter with wetting fluid. For example, the fluid packet can be positioned within the catheter container so that the container must be moved to contact the insertable length of the catheter with wetting fluid.

The present catheter container can have a configuration and can be constructed of material suitable for enclosing a catheter and a fluid packet. The catheter container has a configuration and/or construction suitable for moving the container to move fluid within the container over the length of the catheter. The catheter container can be adapted and configured to be moved in any of a variety of ways including moving the container through space (e.g., rotating, tipping, or waving the container), moving a portion of the container (e.g., bending the container), or manipulating the container (e.g., pushing wetting fluid, material making up the container, or contents of the container). For example, the catheter container can be sufficiently small and light that it can be tipped or twisted to move water over the length of the catheter. For example, the catheter container can be made of flexible material that can be manipulated to move fluid over the length of the catheter.

In an embodiment the catheter container has a configuration of an elongated sheath, pouch, envelope, pocket, or the like. In an embodiment the catheter container, can be formed from material that provides a view of the catheter against an opaque or translucent background. For example, the catheter container can be formed from a sheet of transparent material and a sheet of opaque or translucent material. The sheets can be bonded together around the perimeter of a cavity containing the catheter and the fluid packet. For example, the catheter container can be in the form of a pouch formed from a translucent material and a clear material, a first side of the pouch comprising the translucent material and a second side of the pouch comprising the clear material. Such a pouch can be sealed around the edges.

In an embodiment, the catheter is loose within the catheter container. In an embodiment, the catheter is removably positioned within catheter container. Similarly, in an embodiment, the fluid packet is loose within the catheter container. In an embodiment, the fluid packet is fixedly positioned within the catheter container. In an embodiment, the fluid packet is removably positioned within the catheter container. In an embodiment, the fluid packet is a compartment formed from the material of the catheter container.

The present fluid packet can have a configuration and can be constructed of material suitable for containing fluid in a catheter container. The fluid packet can be configured for containing sufficient fluid to completely wet the catheter with movement of the catheter container, but to only incompletely wet the catheter without movement of the catheter container. The fluid packet can be configured for releasing fluid upon application of a force to the packet without opening the catheter container. For example, the fluid packet can include a fluid filled cavity bounded by two pieces of material bonded with a seam. Squeezing or striking such a fluid packet can breach the material or the seam and release the fluid. The material or seam can be substantially uniform around the edge.

The fluid packet can take any of a variety of forms, such as a sachet, an ampoule, a tube, or the like. The fluid packet can be made of any of a variety of materials such as aluminum foil, poly(vinylidene chloride), metallized film, or the like. The metallized film can be or include metallized poly(ethylene terephthalate). Preferably, the fluid packet is made from a hydrophobic material, such as a hydrophobic plastic, such as polyethylene or material including polyethylene. The fluid packet can include a first piece of packet material and a second piece of packet material. In this configuration, the first piece and second piece of packet material can be sealed to one another around the edges. The seal can be substantially uniform around the edge.

The fluid packet can contain any of a variety of fluids suitable for wetting a catheter. In an embodiment, the fluid is an aqueous wetting composition. The aqueous wetting composition can be any of a variety of aqueous wetting compositions suitable for wetting a catheter, such as a hydrophilic catheter. The aqueous wetting composition can be or include a sterile composition, such as sterile water (e.g. tap, deionized, or distilled water), sterile saline solution, or the like. In an embodiment, the aqueous wetting composition is or is made from distilled water.

The fluid packet can contain a variety of antimicrobial agents in the aqueous wetting composition. For example, the aqueous wetting composition can be an acid in water. One embodiment can be glacial acetic acid in sterile water at a concentration of about 0.1% to about 5%. For example, the aqueous wetting composition can be a quaternary ammonium compound in water. One embodiment can be benzalkonium chloride in sterile water from about 0.02% to about 0.5%. For example, the aqueous wetting composition can be a guanidine or biguanidine derivative in water. One embodiment can be chlorhexidine gluconate in sterile water from about 1% to about 4%. Another embodiment can belyhexamethylene biguanide hydrochloride in sterile water at a concentration of about 1 mg/ml. For example, the aqueous wetting composition can be iodine in water. One embodiment can be iodine in sterile water at about 0.035% or less. Another embodiment can be iodopovidone/PVP-I in sterile water from about 0.5% to about 10%. For example, the aqueous wetting composition can be a nitrofuran compound in water. One embodiment can be nitrofurazone in sterile water at about 0.025% or less. For example, the aqueous wetting composition can be a divalent sulfate in water. One embodiment can be zinc sulfate in sterile water from about 0.05% to about 10%.

In an embodiment, the fluid packet can contain an aqueous composition including lubricant and antimicrobial agent. In an embodiment, the present invention employs an antimicrobial agent soluble in water or saline, and a hydrophilic catheter that becomes lubricated upon contact with water or saline.

The combination of lubricant and antimicrobial in one aqueous composition is a significant improvement over the previous systems for catheter lubrication and sterilization. The fluid packet wetting system eliminates the need for a sponge containing antiseptic, thereby removing unnecessary waste for the user. The removal of the sponge also eliminates the need to perform two independent steps while maintaining a sterile environment, thereby streamlining the events prior to catheter insertion. The fluid packet wetting system eliminates the need to have two independent departments or reservoirs with in the sterile environment, thereby reducing the risk that a reservoir will prematurely rupture. A one packet system improves the number of configurations and options a catheter wetting system can come in. A one packet system reduces the total volume of liquid needed for sterilization and lubrication, thereby decreasing the chances of spilling or leaking during use.

In an embodiment the fluid packet can contain an aqueous composition known as “ionized water,” which is also known as electrolysis reduced water, electrolyzed anode water, reduced water, electrolyzed ionized water, alkaline antioxidant water, or the like.

In an embodiment, the fluid packet is of a volume that provides insufficient fluid for completely wetting the catheter without moving or manipulating the catheter container. For example, the fluid packet can have a volume of about 10-15 mL in a much larger container. Advantageously, such a low-volume fluid packet can provide for less water that might be spilled during use of the catheter and/or disposal of the used container, less wastewater, and/or less mass and volume to package and transport.

The present system can include any of a variety of components useful with the catheter. For example, the added component can be useful for inserting, using, removing, or disposing of the catheter. The present system can include a urine collection bag. The present system can include instructions about the use of the system or any part of the system. Such instructions, for example, can state that the catheter container be moved to move the wetting fluid over the insertable coated length of the catheter. Such instructions can be printed on the catheter container or on an instruction sheet included with the system. An article of manufacture can include the system plus one or more of these additional components.

### The Method

The present invention relates to a method for wetting a catheter. The method can include providing a catheter and a fluid packet in a catheter container, breaching the fluid packet to release fluid into the container thereby only incompletely wetting the catheter, and moving the container to wet the catheter. The method can include providing the system of the invention. In an embodiment, providing includes providing a catheter container with the catheter and the fluid packet sealed inside.

The method can include breaching the fluid packet to release the fluid by any of a variety of mechanisms or conditions suitable for releasing fluid from a fluid packet inside a container, such as a catheter container. Breaching can be accomplished by a health care professional or by a person who will be using the catheter. In an embodiment, breaching can be accomplished by a person with diminished strength, coordination, or manual dexterity. Breaching can include, for example, striking, piercing, or squeezing the fluid packet. Preferably, breaching includes, for example, striking, piercing, or squeezing the fluid packet without opening the catheter container. For example, breaching can include squeezing or striking the fluid packet through material of the catheter container.

Moving the container to wet the catheter can be accomplished through typical ways in the which a person moves or manipulates a small container such as a catheter container. For example, moving the container can include tilting or rotating one end of the catheter container relative to its other end, manipulating the catheter container to move the water, or both moving and manipulating the container. For example, manipulating the container can include applying finger pressure to material forming the catheter container to push fluid within the catheter container. Preferably, moving the catheter container achieves moving the fluid over the insertable coated length of the hydrophilic catheter. Moving the catheter container can be conducted over a period of time sufficient to repeatedly contact each portion of the insertable length of the catheter. In that way, the insertable length can be contacted for a sufficient time to completely wet the catheter. Preferably, breaching the fluid packet and moving the container are conducted without opening the catheter container.

The method can also include other procedures relating to catheters, particularly urinary catheters. For example, the method can include opening the catheter container, removing the wetted catheter from the opened container, and/or using the wetted and removed catheter.

### Illustrated Embodiments

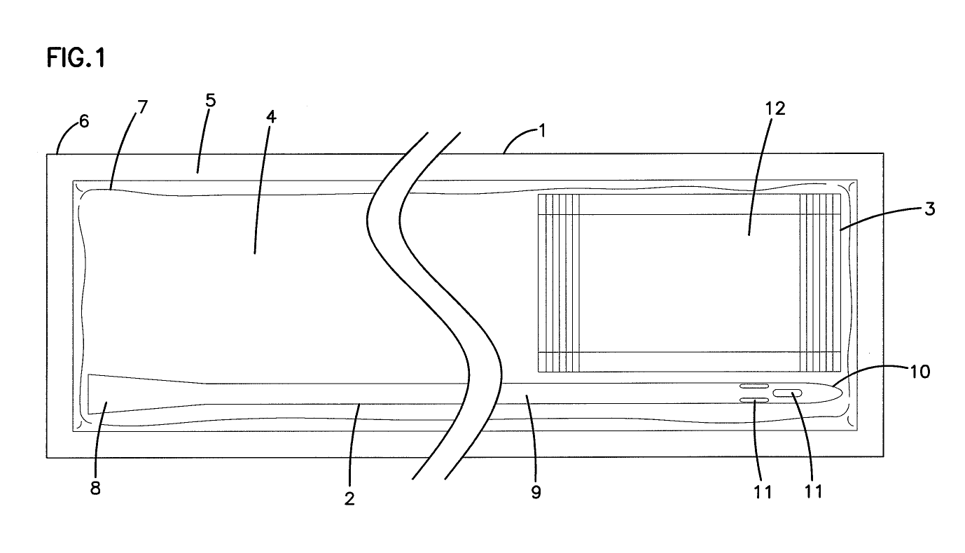

FIG. 1 schematically illustrates an embodiment of a wetting system according to the present invention. The illustrated embodiment of the system includes catheter pouch 1, wettable catheter 2, and fluid sachet 3.

Catheter pouch 1 defines a pouch interior 4 that contains wettable catheter 2 and fluid sachet 3. Pouch interior 4 is bounded by seam 5. In an embodiment, catheter pouch 1 is composed of an opaque or translucent back sheet 6 and transparent cover sheet 7. Back sheet 6 can be composed of a plastic polymer such as polyethylene, e.g., low density polyethylene. Cover sheet 7 can be composed of a plastic polymer such as polyethylene, e.g., high density polyethylene. Back sheet 6 and cover sheet 7 can be joined to form seam 5 by an adhesive, by a weld, or the like. Seam 5, back sheet 6, and cover sheet 7 are preferably liquid tight. Back sheet 6 and cover sheet 7 are preferably permeable to sterilants such as ethylene oxide.

Wettable catheter 2 includes flared outlet 8, shaft 9, tip 10, and eyelet 11. Shaft 9 and tip 10 define a lumen (not shown) leading from eyelet 11 to outlet 8. Tip 10 and a major portion of shaft 9 of wettable catheter 2 are coated with a wettable coating (not shown).

Fluid sachet 3 defines a fluid chamber 12, which can contain fluid for wetting wettable catheter 2. Fluid sachet 3 can contain sterile water, sterile saline, or another fluid suitable for wetting wettable catheter 2. Fluid sachet 3 can be opened, for example, by applying finger or hand pressure or by striking by hand or with a small object. Opening fluid sachet 3 releases substantially all of the fluid it contains into pouch interior 4 without completely wetting shaft 9 of wettable catheter 2. In particular, release of fluid from fluid sachet 3 only incompletely wets the wettable coating. Fluid sachet 3 can be located at any position within pouch interior 4.

In an embodiment, fluid sachet 3 can be made from polyethylene and aluminum foil. Each piece making up fluid sachet 3 can include a layer aluminum foil sandwiched between layers of polyethylene. The polyethylene is adhered to the aluminum foil.

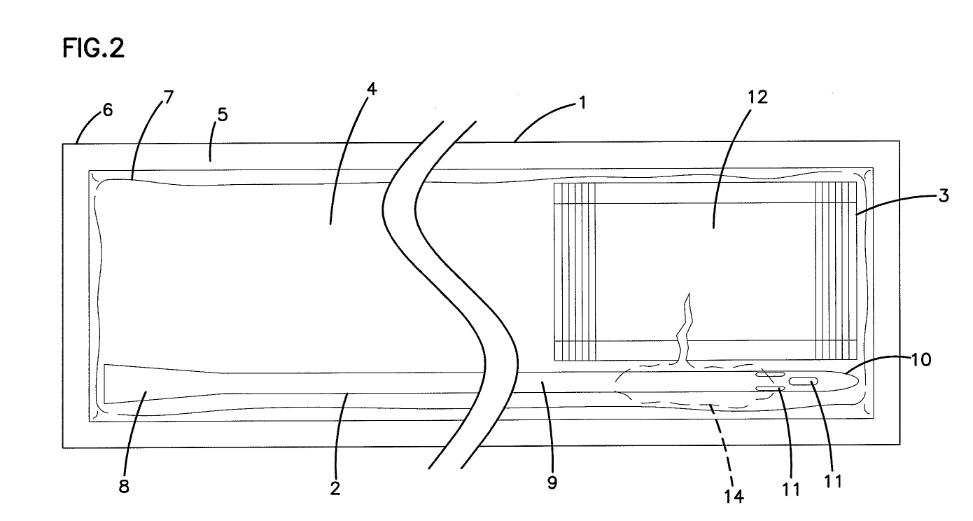

FIG. 2 schematically illustrates an embodiment of the wetting system according to FIG. 1 in which fluid sachet 3 has been opened and fluid 14 has been released from fluid sachet 3. Substantially all of fluid 14 has been released from the fluid sachet 3, but fluid 14 has only incompletely wetted shaft 9 of wettable catheter 2.

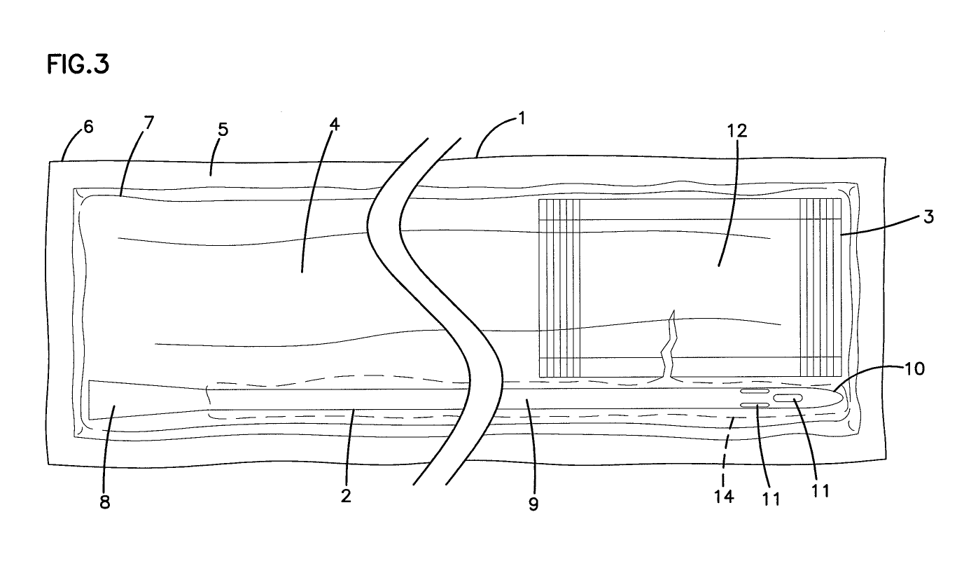

FIG. 3 schematically illustrates an embodiment of the wetting system according to FIGS. 1 and 2 in which catheter pouch 1 has been rocked, manipulated, or otherwise moved to spread fluid 14 along a substantial portion of shaft 9 of wettable catheter 2.

### Segmented Catheter Pouch

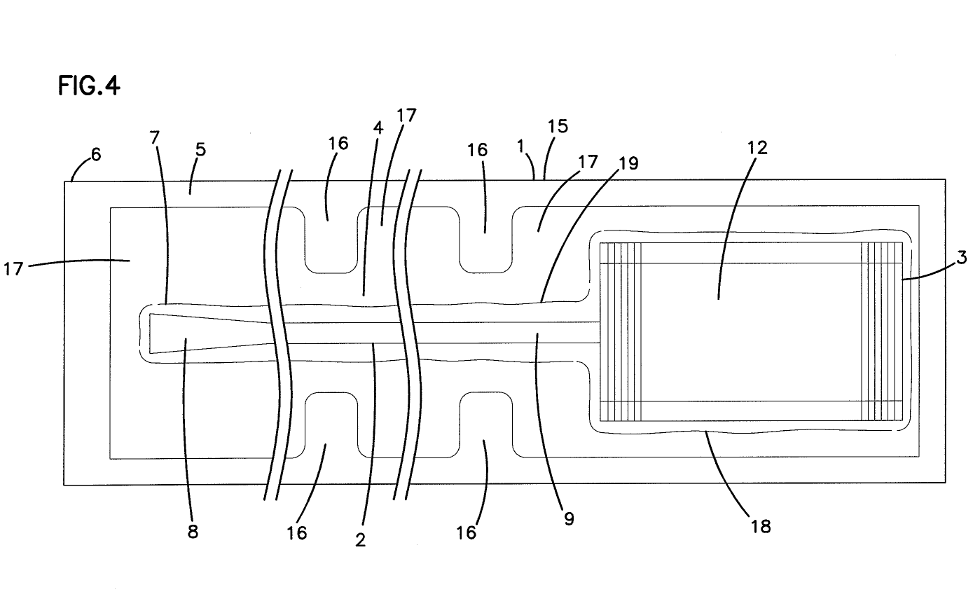

FIG. 4 schematically illustrates an embodiment of the wetting system according to the present invention. The illustrated embodiment of the system includes catheter pouch 1, wettable catheter 2, and fluid sachet 3. In this embodiment, catheter pouch 1 is in the form of segmented catheter pouch 15. Segmented catheter pouch 15 is constructed similarly to catheter pouch 1 and includes the features of catheter pouch 1.

As illustrated in FIG. 4, segmented catheter pouch 15 includes one or more pouch dividers 16. In the illustrated embodiment, pouch dividers 16 come in pairs, each pair being configured to narrow catheter pouch 1 to divide catheter pouch 1 into two or more segments 17. Segmented catheter pouch 15 can include, for example, three pairs of pouch dividers (FIG. 4). Alternatively, a single pouch divider 16 can narrow catheter pouch 1 to divide catheter pouch 1 into two or more segments 17, for example, by extending as the same distance across catheter pouch 1 as a pair of pouch dividers 16. Typically, pouch divider 16 is made up of back sheet 6 and cover sheet 7. Back sheet 6 and cover sheet 7 can be joined to form pouch divider 16 by, for example, an adhesive, a weld, or the like. Pouch divider 16 can be an extension of or protrusion from seam 5.

Segmented catheter pouch 15 can include two, three, four, or more segments 17. Wettable catheter 2 can reside in a plurality of segments 17. Fluid sachet 3 can reside in a single segment 17. In an embodiment, fluid sachet 3 resides in a segment 17 also occupied by tip 10 of wettable catheter 2. In an embodiment, fluid sachet 3 resides in a segment 17 not occupied by tip 10 of wettable catheter 2. In an embodiment, fluid sachet 3 resides in a segment 17 also occupied by shaft 9 of wettable catheter 2. In an embodiment, fluid sachet 3 resides in a segment 17 also occupied by flared outlet 8 of wettable catheter 2.

Segment 17 of segmented catheter pouch 15 that contains fluid sachet 3 can be in the form of a sachet housing 18. Sachet housing 18 is typically of larger volume than other segments to provide space for housing fluid sachet 3. Sachet housing 18 can, for example, can be made up of back sheet 6 and cover sheet 7, with cover sheet defining a larger volume than it does in other segments 17. For example, back sheet 6 and cover sheet 7 can define a rectangular volume for sachet housing 18 (FIG. 4).

Catheter pouch 1 or segmented catheter pouch 15 can include a catheter channel 19. Catheter channel 19 can, for example, can be made up of back sheet 6 and cover sheet 7. Catheter channel 19 is dimensioned for containing wettable catheter 2 and retaining fluid 14 near or on catheter 2.

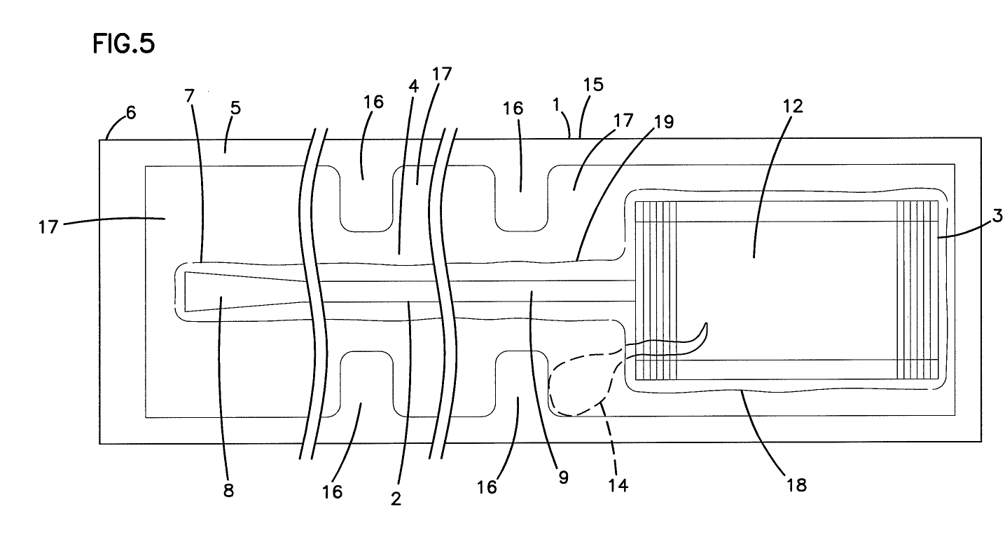

FIG. 5 schematically illustrates an embodiment of the wetting system according to FIG. 4 in which fluid sachet 3 has been opened and fluid 14 has been released from fluid sachet 3. Substantially all of fluid 14 has been released from the fluid sachet 3, but fluid 14 has only incompletely wetted shaft 9 of wettable catheter 2. In an embodiment, pouch dividers 16 restrict the flow of fluid 14 in segmented catheter pouch 15. Pouch dividers 16 can also direct fluid toward wettable catheter 2 in segmented catheter pouch 15.

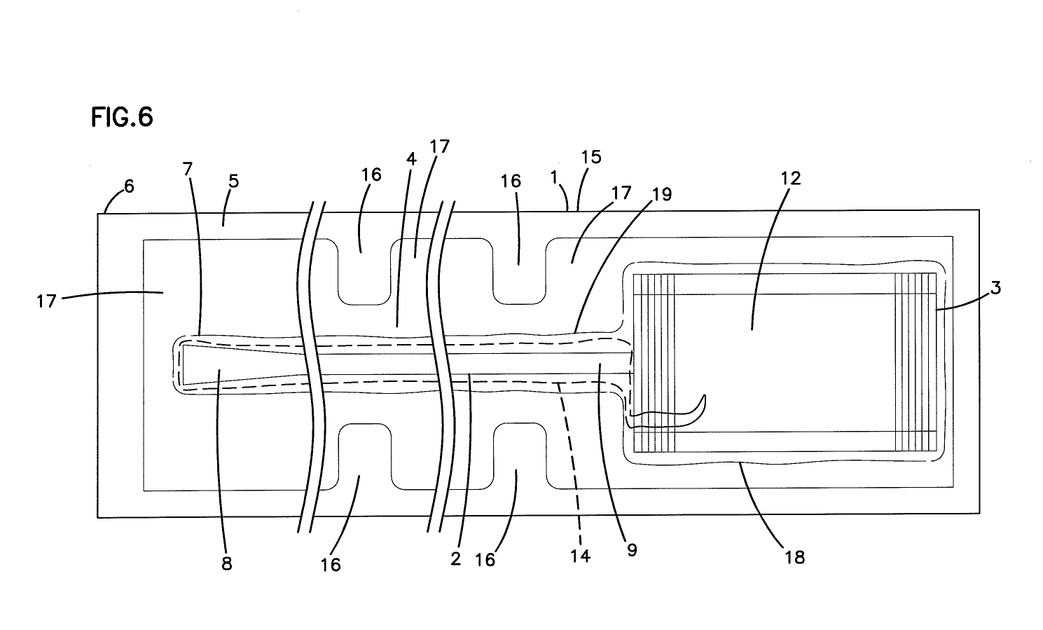

FIG. 6 schematically illustrates an embodiment of the wetting system according to FIGS. 4 and 5 in which segmented catheter pouch 15 has been rocked, manipulated, or otherwise moved to spread fluid 14 along a substantial portion of shaft 9 of wettable catheter 2.

It should be noted that, as used in this specification and the appended claims, the singular forms “a,” “an,” and “the” include plural referents unless the content clearly dictates otherwise. Thus, for example, reference to an apparatus including “a widget” includes a an apparatus including two or more widgets. It should also be noted that the term “or” is generally employed in its sense including “and/or” unless the content clearly dictates otherwise.

It should also be noted that, as used in this specification and the appended claims, the phrase “adapted and configured” describes a system, apparatus, or other structure that is constructed or configured to perform a particular task or adopt a particular configuration. The phrase “adapted and configured” can be used interchangeably with other similar phrases such as arranged and configured, constructed and arranged, adapted, constructed, configured, manufactured and arranged, and the like.

The invention has been described with reference to various specific and preferred embodiments and techniques. However, it should be understood that many variations and modifications may be made while remaining within the spirit and scope of the invention.

We claim:

1. A catheter wetting system comprising:
a catheter container;
a fluid packet; and
a hydrophilic catheter;
the fluid packet and the hydrophilic catheter being sealed within the catheter container;
the fluid packet containing an aqueous composition comprising antimicrobial agent;
the fluid packet and catheter container being adapted and configured so that opening the packet only incompletely wets the coated length of the hydrophilic catheter.

2. The catheter wetting system of claim 1, wherein the catheter container is adapted and configured to be moved to move wetting fluid over the insertable coated length of the hydrophilic catheter.

3. The catheter wetting system of claim 1, wherein the catheter container comprises flexible material that can be manipulated to spread wetting fluid over the coated length of the hydrophilic catheter.

4. The catheter wetting system of claim 1, wherein the catheter container comprises a pouch formed from a translucent material and a clear material, a first side of the pouch comprising the translucent material and a second side of the pouch comprising the clear material, the pouch being sealed around the edges.

5. The catheter wetting system of claim 1, wherein the fluid packet comprises a first piece of packet material and a second piece of packet material, the first piece and second piece of packet material being sealed to one another around the edges, the seal being substantially uniform around the edge.

6. The catheter wetting system of claim 1, wherein the fluid packet comprises hydrophobic material.

7. The catheter wetting system of claim 6, wherein the hydrophobic material comprises polyethylene and aluminum foil.

8. The catheter wetting system of claim 1, wherein the fluid packet comprises an aqueous wetting composition.

9. The catheter wetting system of claim 8, wherein the aqueous wetting composition comprises sterile water or sterile saline solution.

10. The catheter wetting system of claim 8, wherein the sterile water or sterile saline solution comprise sterile distilled water.

11. The catheter wetting system of claim 1, wherein the hydrophilic catheter comprises a hydrophilic coating over an insertable length of the catheter.

12. The catheter wetting system of claim 1, further comprising a urine collection bag.

13. The catheter wetting system of claim 1, further comprising instructions that the catheter container be moved to move the wetting fluid over the insertable coated length of the hydrophilic catheter.

14. The catheter wetting system of claim 1, further comprising instructions printed on the catheter container stating that the catheter container be moved to move the wetting fluid over the insertable coated length of the hydrophilic catheter.

15. The catheter wetting system of claim 1, wherein the antimicrobial agent is water soluble.

16. The catheter wetting system of claim 1, wherein the aqueous composition comprises sterile water or saline.
